(19) United States
(12) Patent Application Publication
(10) Pub. No.: US 2026/0224279 A1
(43) Pub. Date: Aug. 6, 2026

(54) SKIN STIMULATION DEVICE

(71) Applicant: SSB MEDICAL COOPERATION, Seoul (KR)

(72) Inventors: Suk Bae SEO, Seoul (KR); Ja Young KIM, Seoul (KR); Ho Joon SEO, Seoul (KR)

(73) Assignee: SSB MEDICAL COOPERATION, Seoul (KR)

(21) Appl. No.: 19/576,418

(22) Filed: Mar. 24, 2026

Related U.S. Application Data
(63) Continuation of application No. PCT/KR2024/014444, filed on Sep. 25, 2024.

(30) Foreign Application Priority Data
Sep. 25, 2023 (KR) 10-2023-0128549

(51) Int. Cl. A61B 18/14; A61B 18/00; A61B 18/06; A61B 18/12

(52) U.S. Cl. CPC A61B 18/1477; A61B 18/06; A61B 18/1206; A61B 2018/00184; A61B 2018/00452; A61B 2018/0072; A61B 2018/00744; A61B 2018/068; A61B 2018/143

(57) ABSTRACT

The present disclosure relates to a skin stimulation device including one or more cannulas inserted into or discharged from a target site of skin; a hydrogen gas supply part for supplying hydrogen gas into the cannulas; a current supply part for supplying a radio-frequency current to the cannulas; and a processor configured to control the current supply part and the hydrogen gas supply part, wherein the processor operates the hydrogen gas supply part and the current supply part when the cannulas are inserted into the target site to induce tissue damage of the target site.

### CROSS-REFERENCE TO RELATED APPLICATIONS

The present application is a continuation of International Patent Application No. PCT/KR 2024/014444, filed on Sep. 25, 2024, which is based upon and claims the benefit of priority to Korean Patent Application No. 10-2023-0128549 filed on Sep. 25, 2023. The disclosures of the above-listed applications are hereby incorporated by reference herein in their entirety.

### BACKGROUND

### 1. Technical Field

The present disclosure relates to a skin stimulation device.

### 2. Description of Related Art

In general, a skin stimulation device that performs thermal stimulation by supply of electrical energy inserts a needle into a target site of skin and applies a radio-frequency current to the needle, such that the radio-frequency current is delivered to the target site of the skin through the needle.

The radio-frequency current delivered to the target site of the skin through the needle is converted into thermal energy that instantaneously increases a temperature of the target site of the skin, thereby inducing coagulative necrosis due to tissue damage of the target site of the skin. The skin tissue subjected to the coagulative necrosis can be naturally restored by a damage recovery mechanism of a living body.

However, since a conventional skin stimulation device supplies only thermal energy by delivery of a radio-frequency current to a target of the skin, there is a problem in that efficiency of tissue damage of the target site of the skin is reduced.

### SUMMARY

The present disclosure has been devised to solve the above-described problem, and an object of the present disclosure is to provide a skin stimulation device capable of improving efficiency of tissue damage of a target site of skin by simultaneously performing tissue damage by hydrogen explosion and tissue damage by thermal energy at the target site of the skin.

The problems to be solved by the present disclosure are not limited to the problems mentioned above, and other problems not mentioned will be clearly understood by those skilled in the art from the following description.

A skin stimulation device according to an embodiment of the present disclosure may include one or more cannulas inserted into or discharged from a target site of skin; a hydrogen gas supply part for supplying hydrogen gas into the cannulas; a current supply part for supplying a radio-frequency current to the cannulas; and a processor configured to control the current supply part and the hydrogen gas supply part, wherein the processor may induce tissue damage of the target site by operating the hydrogen gas supply part and the current supply part when the cannulas are inserted into the target site.

In addition, the processor may operate the hydrogen gas supply part and then operate the current supply part in a state in which the cannulas are inserted into the target site.

In addition, when the hydrogen gas supply part is operated, the hydrogen gas is injected into the target site, and when the current supply part is operated, the hydrogen gas injected into the target site explodes by application of the radio-frequency current and may induce first tissue damage of the target site, and at the same time, thermal energy generated by the radio-frequency current delivered to the target site may induce second tissue damage of the target site.

In addition, a channel through which the hydrogen gas supplied from the hydrogen gas supply part flows is formed inside the cannulas, and a discharge part through which the hydrogen gas flowing through the channel is discharged may be formed on a circumferential surface of the cannulas.

In addition, the skin stimulation device may further include a cylinder; a plunger through which the one or more cannulas pass and provided to be movable in the cylinder in a first direction away from the skin or in a second direction closer to the skin; and a driving part for moving the plunger in the first direction or the second direction.

In addition, the skin stimulation device may further include a cannula check valve provided in the cannulas, preventing backflow of the hydrogen gas in the cannulas and backflow due to explosion of the hydrogen gas at the target site, and causing the hydrogen gas supplied to the cannulas to flow only from the cannulas to the target site.

In addition, the cannulas may include one or more first cannulas arranged at intervals on one side of the plunger; and one or more second cannulas arranged at intervals on the other side of the plunger, wherein each of the first cannulas may include a first discharge part formed on one surface facing the second cannulas, and each of the second cannulas may include a second discharge part formed on one surface facing the first cannulas.

In addition, the skin stimulation device may further include a cylinder in which the plunger is movably embedded in the first direction or the second direction; and a slip member coupled to an outer circumference of the plunger.

In addition, the skin stimulation device may further include a stopper installed in the cylinder and limiting a movement range of the plunger.

In addition, the skin stimulation device may further include a vortex part formed in a spiral shape along an inner circumferential surface of the cannulas.

Other specific matters of the present disclosure are included in the detailed description and the drawings.

### BRIEF DESCRIPTION OF THE FIGURES

FIG. 1 is a schematic view illustrating a skin stimulation device according to an embodiment of the present disclosure.

FIG. 2 to FIG. 4 are schematic views illustrating an operation process of a skin stimulation device according to an embodiment of the present disclosure.

FIG. 5 to FIG. 7 are schematic views illustrating various embodiments of a skin stimulation device according to an embodiment of the present disclosure.

FIG. 8 is a cross-sectional view illustrating an example of a cannula of a skin stimulation device according to an embodiment of the present disclosure.

### DETAILED DESCRIPTION

Advantages and features of the present disclosure, and methods for achieving the same, will be clearly understood with reference to embodiments described in detail below together with the accompanying drawings. However, the present disclosure is not limited to the embodiments disclosed below and may be implemented in various different forms, and these embodiments are provided only to make the disclosure of the present disclosure complete and to fully inform those skilled in the art to which the present disclosure pertains of the scope of the present disclosure, and the present disclosure is defined only by the scope of the claims.

Terms used in the present specification are for explaining the embodiments and are not intended to limit the present disclosure. In the present specification, a singular form includes a plural form unless specifically stated otherwise in a phrase. The terms “include” and/or “including” used in the specification do not exclude the presence or addition of one or more other components in addition to the mentioned components. Throughout the specification, the same reference numerals refer to the same components, and “and/or” includes each of the mentioned components and all combinations of one or more thereof. Although terms such as “first” and “second” are used to describe various components, these components are not limited by these terms. These terms are used only to distinguish one component from another component. Accordingly, a first component mentioned below may also be a second component within the technical idea of the present disclosure.

Unless otherwise defined, all terms used in the present specification, including technical and scientific terms, may be used with meanings commonly understood by those skilled in the art to which the present disclosure pertains. In addition, terms defined in generally used dictionaries are not to be interpreted ideally or excessively unless clearly specifically defined.

Hereinafter, embodiments of the present disclosure will be described in detail with reference to the accompanying drawings.

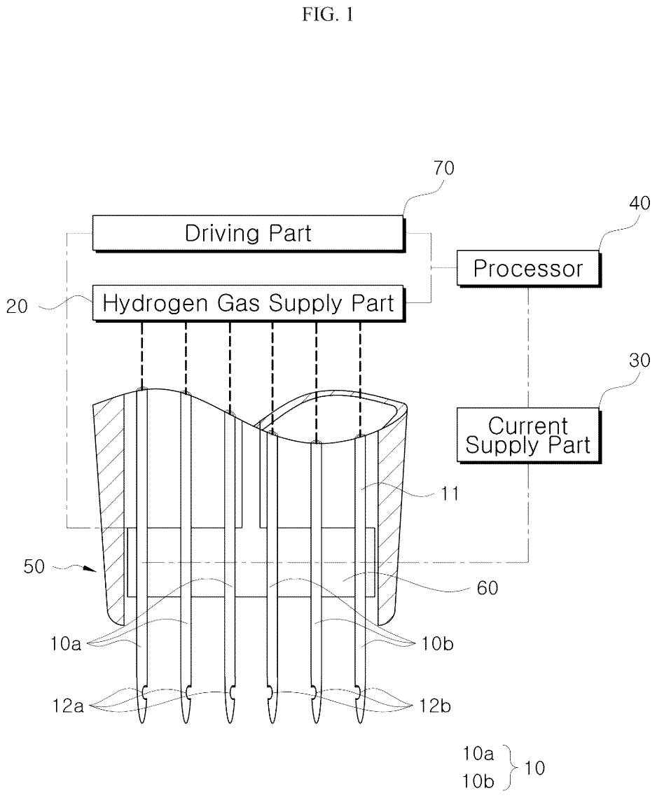

FIG. 1 is a schematic view illustrating a skin stimulation device according to an embodiment of the present disclosure.

As shown in FIG. 1, a skin stimulation device according to an embodiment of the present disclosure may include cannulas 10a and 10b, a hydrogen gas supply part 20, a current supply part 30, and a processor 40.

The cannulas 10a and 10b may be inserted into a target site of skin 1. A channel 11 through which hydrogen gas supplied from the hydrogen gas supply part 20 described later flows may be formed inside the cannulas 10a and 10b, and discharge parts 12a and 12b through which the hydrogen gas flowing through the channel 11 is discharged may be formed on a circumferential surface of the cannulas 10a and 10b.

As an example, the cannulas 10a and 10b may be configured as one or more, and specifically may be configured as a single or a plurality.

As another example, distal ends of the cannulas 10a and 10b may have a sharp shape. Accordingly, the distal ends of the cannulas 10a and 10b may be inserted into a target site of skin 1 to form insertion holes in the target site of the skin 1.

The hydrogen gas supply part 20 serves to supply hydrogen into the cannulas 10a and 10b, and here, an inside of the cannulas 10a and 10b may be the channel 11 of the cannulas 10a and 10b. The hydrogen gas supply part 20 may supply hydrogen into the cannulas 10a and 10b under control of the processor 40.

The hydrogen gas supply part 20 may include a hydrogen gas tank (not shown) a hydrogen gas pipe (not shown), and a hydrogen gas check valve (not shown). Here, the hydrogen gas tank may store hydrogen gas. In addition, the hydrogen gas pipe may connect the hydrogen gas tank and the channel 11 of the cannulas 10a and 10b, and the hydrogen gas check valve may open or close the hydrogen gas pipe under control of the processor 40 and may serve as a one-way valve allowing the hydrogen gas stored in the hydrogen gas tank to flow only to the channel 11 of the cannulas 10a and 10b. Accordingly, when the hydrogen gas check valve opens the hydrogen gas pipe, the hydrogen gas stored in the hydrogen gas tank may be supplied to the channel 11 of the cannulas 10a and 10b through the hydrogen gas pipe. In addition, when the hydrogen gas check valve closes the hydrogen gas pipe, the hydrogen gas stored in the hydrogen gas tank may not be supplied or may be stopped from being supplied to the channel 11 of the cannulas 10a and 10b through the hydrogen gas pipe.

The current supply part 30 may supply a radio-frequency current to the cannulas 10a and 10b. The current supply part 30 may receive power from a power supply part. Here, the power supply part may be a battery.

The processor 40 serves to control the current supply part 30 and the hydrogen gas supply part 20. The processor 40 may operate the hydrogen gas supply part 20 and the current supply part 30 in a state in which the cannulas 10a and 10b are inserted into a target site of skin 1 to induce a plurality of tissue damages in the target site of the skin 1.

Specifically, the processor 40 may operate the hydrogen gas supply part 20 and then operate the current supply part 30 in a state in which the cannulas 10a and 10b are inserted into the target site. In other words, the processor 40 may sequentially operate the hydrogen gas supply part 20 and the current supply part 30 in a state in which the cannulas 10a and 10b are inserted into the target site. Accordingly, when the hydrogen gas supply part 20 is operated, hydrogen gas may be injected into the target site of the skin 1. Thereafter, when the current supply part 30 is operated, the hydrogen gas injected into the target site of the skin 1 explodes by application of a radio-frequency current and induces first tissue damage of the target site of the skin 1, and at the same time, thermal energy generated by the radio-frequency current delivered to the target site induces second tissue damage of the target site, and as a result, since the first tissue damage and the second tissue damage are simultaneously induced in the target site of the skin 1, efficiency of tissue damage of the target site of the skin 1 can be improved. Here, the first tissue damage of the target site of the skin 1 may occur due to a physical impact caused by explosion of hydrogen gas. In addition, the second tissue damage of the target site of the skin 1 may occur due to a temperature increase of the target site of the skin 1 caused by thermal energy generated by the radio-frequency current.

Meanwhile, the skin stimulation device according to an embodiment of the present disclosure may further include a cylinder 50, a plunger 60, and a driving part 70.

The cylinder 50 may serve as a main body of the present disclosure. A handpiece may be used instead of the cylinder 50. As an example, the cylinder 50 may be disposed to face a target site of skin 1. As another example, the cylinder 50 may be in close contact with the target site of the skin 1.

The plunger 60 is provided such that the one or more cannulas 10a and 10b pass therethrough and is provided to be movable in the cylinder 50 in a first direction away from the skin 1 or in a second direction closer to the skin 1.

The driving part 70 may serve to move the plunger 60 in the first direction or the second direction. As an example, the driving part 70 may be an actuator.

In addition, the skin stimulation device according to an embodiment of the present disclosure may further include a cannula check valve (not shown).

The cannula check valve is provided in the cannulas 10a and 10b, prevents backflow of hydrogen gas in the cannulas 10a and 10b and backflow due to explosion of hydrogen gas at the target site of the skin, and allows the hydrogen gas supplied to the cannulas 10a and 10b to flow only from the cannulas 10a and 10b to the target site of the skin.

As an example, the cannula check valve may be disposed at the discharge parts 12a and 12b of the cannulas 10a and 10b.

Hereinafter, an operation process of a skin stimulation device according to an embodiment of the present disclosure will be described. Here, the operation process may be performed by the processor 40 or a practitioner.

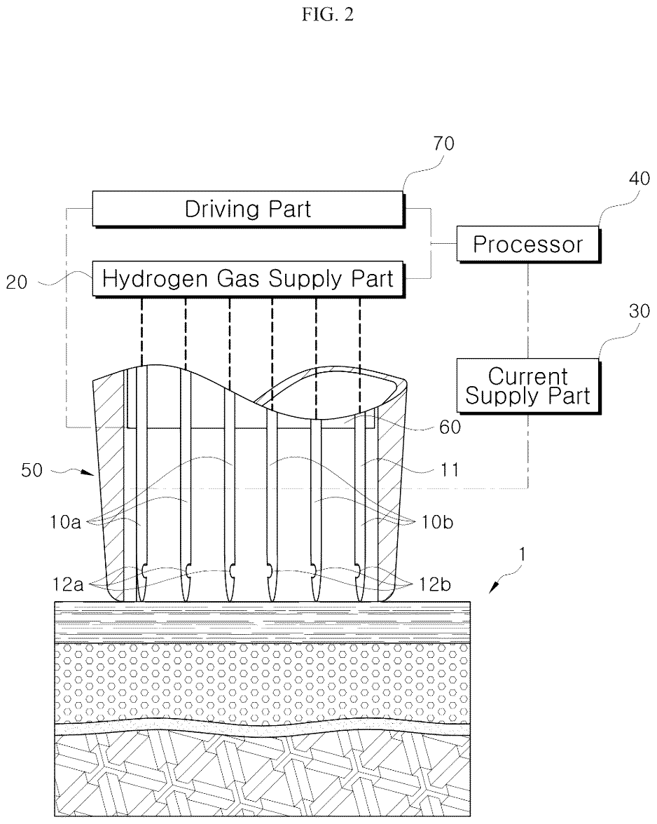

FIG. 2 to FIG. 4 are schematic views illustrating an operation process of a skin stimulation device according to an embodiment of the present disclosure.

First, in a state in which distal ends of the cannulas 10a and 10b are disposed to face skin 1, the cylinder 50 is brought into close contact with a target site of the skin 1 (refer to FIG. 2).

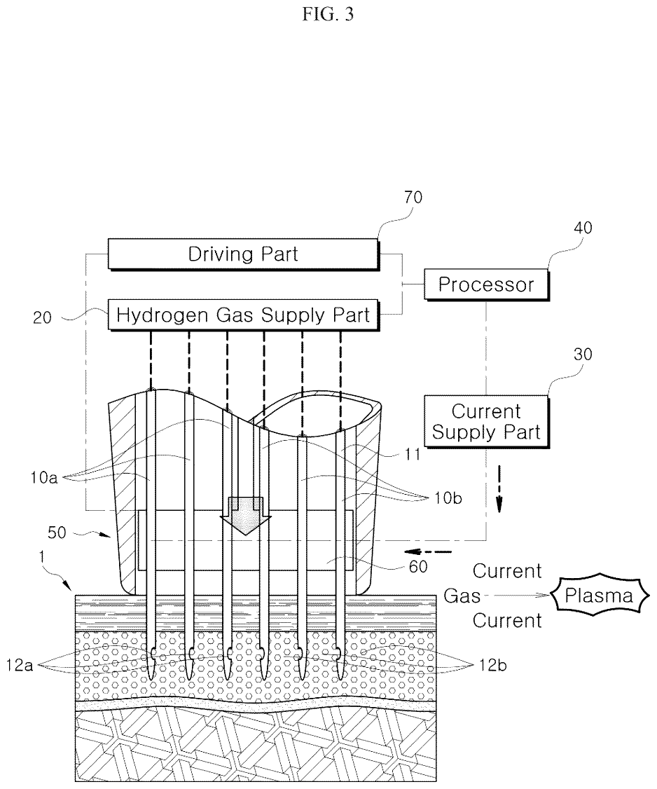

Next, the driving part 70 moves the plunger 60 in a second direction closer to the skin 1. As a result, the cannulas 10a and 10b are inserted into the target site of the skin 1 to form insertion holes in the target site of the skin 1 (refer to FIG. 3).

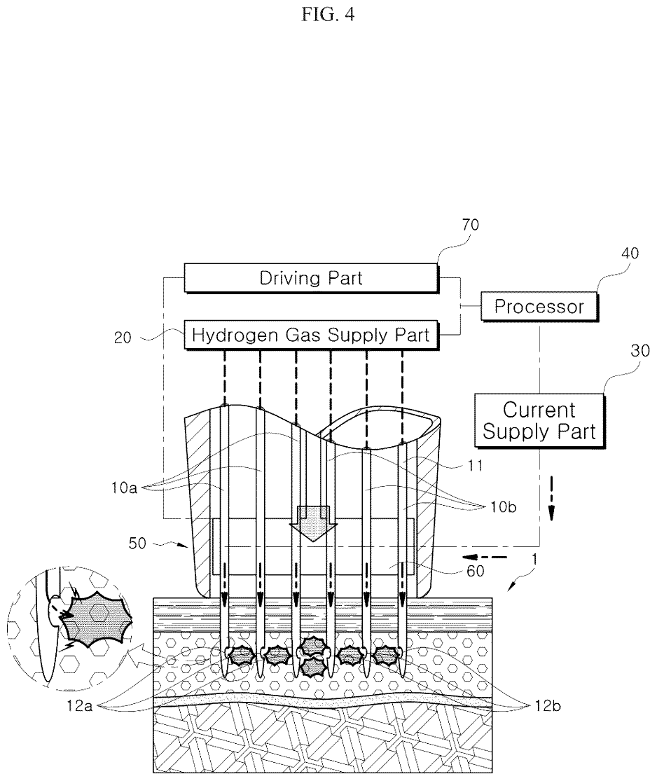

Subsequently, the hydrogen gas supply part 20 and the current supply part 30 are sequentially operated. Accordingly, when the hydrogen gas supply part 20 is operated, hydrogen gas may be injected into the target site of the skin 1. Thereafter, when the current supply part 30 is operated, the hydrogen gas injected into the target site of the skin 1 explodes by application of a radio-frequency current and may induce first tissue damage of the target site of the skin 1, and at the same time, thermal energy generated by the radio-frequency current delivered to the target site may induce second tissue damage of the target site. As a result, since the first tissue damage and the second tissue damage are simultaneously induced in the target site of the skin 1, efficiency of tissue damage of the target site of the skin 1 can be improved (refer to FIG. 4).

As an example, the cannulas may include one or more first cannulas and one or more second cannulas.

The one or more first cannulas 10a may be arranged at intervals on one side of the plunger 60.

The one or more second cannulas 10b may be arranged at intervals on the other side of the plunger 60.

Here, each of the first cannulas 10a may include a first discharge part 12a formed on one surface facing the second cannulas 10b, and each of the second cannulas 10b may include the second discharge part 12b formed on one surface facing the first cannulas 10a. Accordingly, in a state in which the one or more first cannulas 10a and the one or more second cannulas 10b are inserted into the target site of the skin 1, a relatively large amount of hydrogen gas may be concentrated in a region of the target site of the skin 1 between the first discharge part 12a of the first cannula 10a and the second discharge part 12b of the second cannula 10b adjacent to each other. Thereafter, when the current supply part 30 is operated, relatively large tissue damage may occur in the region of the target site of the skin 1 between the first discharge part 12a of the first cannula 10a and the second discharge part 12b of the second cannula 10b adjacent to each other. Accordingly, relatively large tissue damage may be generated in a portion requiring relatively large tissue damage in the target site of the skin 1.

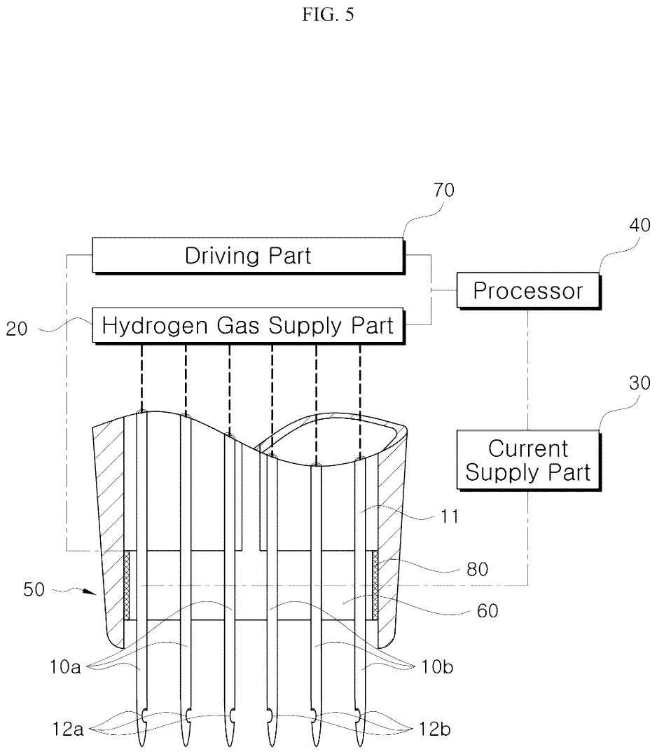

FIG. 5 to FIG. 7 are schematic views illustrating various embodiments of a skin stimulation device according to an embodiment of the present disclosure.

As shown in FIG. 5, the skin stimulation device according to an embodiment of the present disclosure may further include a slip member 80.

The slip member 80 may be coupled to an outer circumference of the plunger 60. The slip member 80 may serve to seal between the cylinder 50 and the plunger 60. In addition, when the plunger 60 moves in the first direction or the second direction, the slip member 80 may slip along the cylinder 50 and may also serve to reduce friction between the plunger 60 and the cylinder 50.

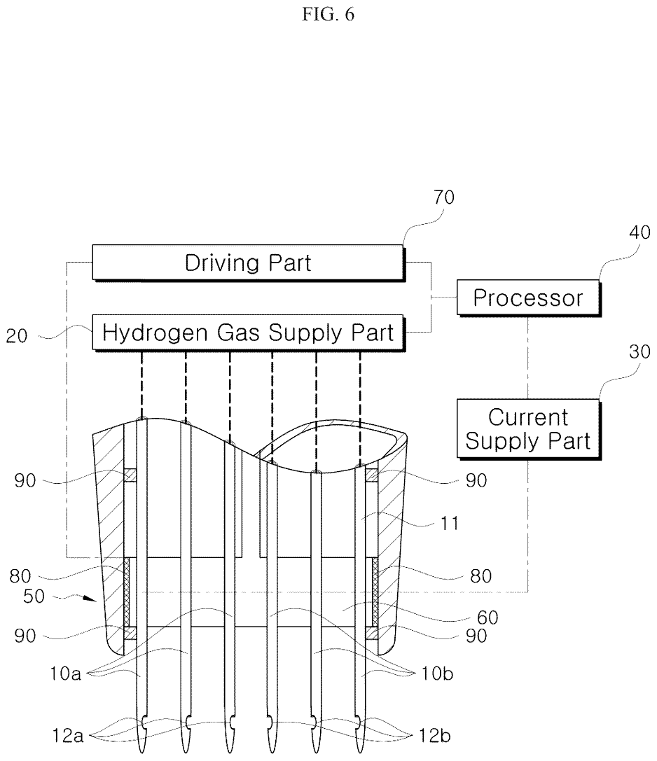

As shown in FIG. 6, the skin stimulation device according to an embodiment of the present disclosure may further include a stopper 90.

The stopper 90 may be provided in the cylinder 50 and may limit a movement range of the plunger 60. The stopper 90 may be provided in plurality, and one of the plurality of stoppers 90 may be installed at an upper portion of the cylinder 50 and one of the plurality of stoppers 90 may be installed at a lower portion of the cylinder 50, thereby limiting the movement range of the plunger 60.

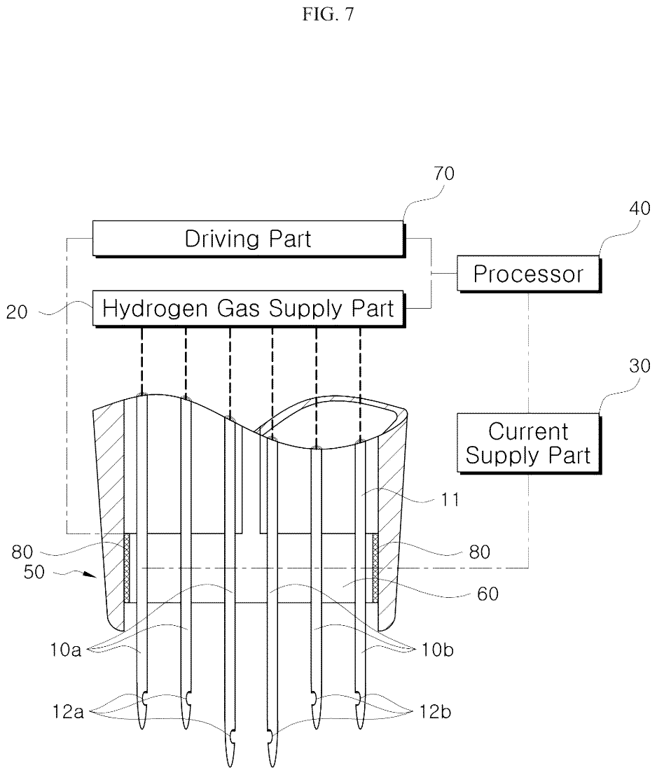

Meanwhile, when the plurality of cannulas 10a and 10b are inserted into the target site of the skin 1, a surface of the skin 1 may be in a deflected state due to pressure applied to the surface of the skin 1 by the plurality of cannulas 10a and 10b, and the cannulas 10a and 10b disposed at a center of the plunger 60 among the plurality of cannulas 10a and 10b is inserted into the skin 1 in a more lowered state than the cannula 10a and 10b disposed at an edge of the plunger 60. To compensate for this, it is preferable that lengths by which the plurality of cannulas 10a and 10b protrude from the plunger 60 are different from each other. For this, among the plurality of cannulas 10a and 10b, the cannulas 10a and 10b disposed at an edge of the plunger 60 may protrude from the plunger 60 by a smaller length than the cannulas 10a and 10b disposed at a center of the plunger 60. Accordingly, when the plurality of cannulas 10a and 10b are inserted into the target site of the skin 1, a deflected state of the surface of the skin 1 due to pressure applied by the plurality of cannulas 10a and 10b can be alleviated (refer to FIG. 7).

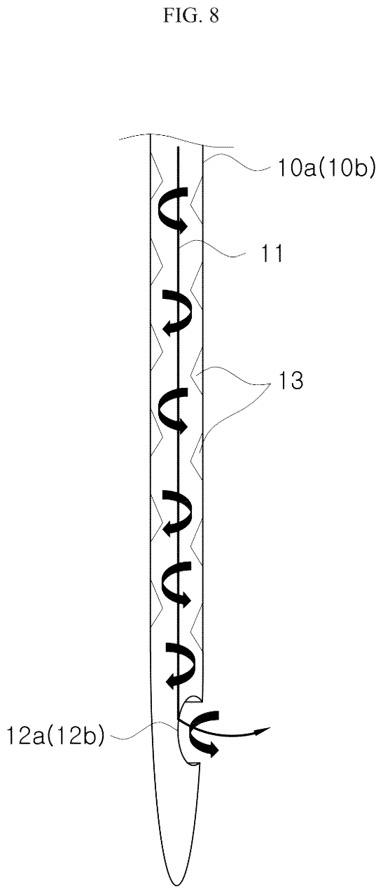

FIG. 8 is a cross-sectional view illustrating an example of a cannula of a skin stimulation device according to an embodiment of the present disclosure.

As shown in FIG. 8, the skin stimulation device according to an embodiment of the present disclosure may further include a vortex part 13.

The vortex part 13 may be formed in a spiral shape along an inner circumferential surface of the cannulas 10a and 10b. The vortex part 13 may form a vortex in hydrogen gas flowing along the cannulas 10a and 10b. As such, when the hydrogen gas in which the vortex is formed is discharged through the discharge parts 12a and 12b of the cannulas 10a and 10b, the hydrogen gas may spread to a wider area of the target site of the skin 1.

According to the present disclosure, the skin stimulation device according to an embodiment of the present disclosure can improve efficiency of tissue damage of a target site of the skin 1 by simultaneously performing tissue damage by hydrogen explosion and tissue damage by thermal energy at the target site of the skin 1.

Although embodiments of the present disclosure have been described above with reference to the accompanying drawings, those skilled in the art to which the present disclosure pertains will understand that the present disclosure may be implemented in other specific forms without changing the technical idea or essential features thereof. Therefore, it should be understood that the embodiments described above are illustrative in all respects and are not restrictive.

A skin stimulation device according to an embodiment of the present disclosure can improve efficiency of tissue damage of a target site of skin by simultaneously performing tissue damage by hydrogen explosion and tissue damage by thermal energy at the target site of the skin.

Effects of the present disclosure are not limited to the above-mentioned effects, and other effects not mentioned above will be clearly understood by those skilled in the art from the description.

What is claimed is:

1. A skin stimulation device, comprising:
one or more cannulas inserted into or discharged from a target site of skin;
a hydrogen gas supply part for supplying hydrogen gas into the cannulas;
a current supply part for supplying a radio-frequency current to the cannulas; and
a processor is configured to control the current supply part and the hydrogen gas supply part,
wherein the processor induces tissue damage of the target site by operating the hydrogen gas supply part and the current supply part when the cannulas are inserted into the target site.

2. The skin stimulation device according to claim 1,
wherein the processor is configured to operate the hydrogen gas supply part and then operates the current supply part in a state in which the cannulas are inserted into the target site.

3. The skin stimulation device according to claim 2,
wherein, when the hydrogen gas supply part is operated, the hydrogen gas is injected into the target site, and
when the current supply part is operated, the hydrogen gas injected into the target site explodes by application of the radio-frequency current and induces first tissue damage of the target site, and at the same time, thermal energy generated by the radio-frequency current transmitted to the target site induces second tissue damage of the target site.

4. The skin stimulation device according to claim 1,
wherein a channel through which the hydrogen gas supplied from the hydrogen gas supply part flows is formed inside the cannulas, and
a discharge part through which the hydrogen gas flowing through the channel is discharged is formed on a circumferential surface of the cannulas.

5. The skin stimulation device according to claim 4, further comprising:
a cylinder;
a plunger through which the one or more cannulas pass and provided to be movable in the cylinder in a first direction away from the skin or in a second direction closer to the skin; and
a driving part for moving the plunger in the first direction or the second direction.

6. The skin stimulation device according to claim 1, further comprising:
a cannula check valve provided in the cannulas, preventing backflow of the hydrogen gas in the cannulas and backflow due to explosion of the hydrogen gas at the target site, and causing the hydrogen gas supplied to the cannulas to flow only from the cannulas to the target site.

7. The skin stimulation device according to claim 5,
wherein the cannulas comprise:
one or more first cannulas arranged at intervals on one side of the plunger; and
one or more second cannulas arranged at intervals on the other side of the plunger,
wherein each of the first cannulas comprises a first discharge part formed on one surface facing the second cannulas, and
each of the second cannulas comprises a second discharge part formed on one surface facing the first cannulas.

8. The skin stimulation device according to claim 5, further comprising:
a cylinder in which the plunger is movably embedded in the first direction or the second direction; and
a slip member coupled to an outer circumference of the plunger.

9. The skin stimulation device according to claim 8, further comprising:
a stopper installed in the cylinder and limiting a movement range of the plunger.

10. The skin stimulation device according to claim 1, further comprising:
a vortex part formed in a spiral shape along an inner circumferential surface of the cannulas.
